(19) United States
(12) Patent Application Publication
(10) Pub. No.: US 2014/0004189 A1
(43) Pub. Date: Jan. 2, 2014

(54) MODIFIED RELEASE PHARMACEUTICAL COMPOSITIONS MEMANTINE

(71) Applicants: Sunilendu Bhushan Roy, Gujarat (IN); Sushrut Krishnaji Kulkarni, Gujarat (IN); Sandhya Rajendra Shenoy, Gujarat (IN); Imran Shakoor Ghogari, Gujarat (IN); Vijaykumar Shivajrao Patil, Gujarat (IN)

(72) Inventors: Sunilendu Bhushan Roy, Gujarat (IN); Sushrut Krishnaji Kulkarni, Gujarat (IN); Sandhya Rajendra Shenoy, Gujarat (IN); Imran Shakoor Ghogari, Gujarat (IN); Vijaykumar Shivajrao Patil, Gujarat (IN)

(73) Assignee: CADILA HEALTHCARE LIMITED, Ahmedabad (IN)

(21) Appl. No.: 13/976,570

(22) PCT Filed: Jan. 25, 2012
(86) PCT No.: PCT/IN2012/000054

§ 371 Date: Sep. 18, 2013

(30) Foreign Application Priority Data
Jan. 25, 2011 (IN) 220/MUM/2011

(51) Int. Cl. A61K 31/13; A61K 9/48; A61K 9/28

(52) U.S. Cl. CPC A61K 31/13; A61K 9/28; A61K 9/4808
USPC 424/459; 514/662; 424/471; 427/2.21

(57) ABSTRACT

Modified release pharmaceutical compositions of memantine or pharmaceutically acceptable salts thereof are described. The compositions of invention are stable, possess improved formulation characteristics and also provide extended therapeutically effective plasma levels over a twenty four hours period. Processes of making these compositions are also described.

### FIELD OF THE INVENTION

The present invention relates to modified release pharmaceutical compositions of memantine or pharmaceutically acceptable salts thereof. In particular, the compositions of invention are stable, possessing improved formulation characteristics and also provide extended therapeutically effective plasma levels over twenty four hours period. The invention also relates to processes of making such compositions.

### BACKGROUND OF THE INVENTION

Memantine is an orally active NMDA (N-methyl-D-aspartate) receptor antagonist which acts by blocking the NMDA receptors in the brain. It blocks the excessive activity of glutamate, but still allows the normal activation of these receptors that occurs when the brain forms a memory. Therefore it improves the brain functioning in Alzheimer's disease, and may also block the glutamate activity that could cause further damage to the brain cells. It has been also hypothesized that memantine may not only be effective for the treatment of Alzheimer's disease (as well as Parkinson's and other neurological diseases), but may also be effective for the treatment of autism, Attention-Deficit/Hyperactivity Disorder (ADHD) and other autistic spectrum disorders. Memantine has the chemical name 3,5-dimethyladamantan-1-amine.

Memantine hydrochloride is commercially available in the market in products sold under the trademark Namenda®. It is available for oral administration as capsule shaped film-coated tablets containing 5 mg and 10 mg of memantine hydrochloride. Currently, a dosing regimen of memantine twice a day is employed using immediate release tablets and once a day dosing regimen employed using sustained release tablets.

Immediate release regimen is not optimal because patient compliance decreases as the frequency of taking the drug increases. Moreover, after oral administration, memantine is completely absorbed (absolute bioavailability of approximately 100%). For pain treatment, it is very important to maintain the pain relief without additional discomfort. Thus, administration of an immediate-release tablet can lead to greater frequency of adverse pharmacological events due to the fast rate of absorption. Current guidelines for use of memantine in the treatment of Alzheimer's Disease recommends that memantine be administered as a starting dose of 5 mg/day and escalated to the 20 mg/day dose by weekly increases in the dose by 5 mg.

There is therefore an existing and continual need for a once a day modified release formulation containing memantine or its a pharmaceutically acceptable salt with reliable slower absorption over a targeted period of time which may address some of the concerns associated with the use of memantine.

U.S. Pat. No. 3,391,142 discloses memantine and its related compounds, along with their pharmaceutically acceptable salts.

U.S. Pat. No. 5,061,703 discloses method for the prevention or treatment of cerebral ischemia using memantine or salts thereof.

U.S. Pat. No. 5,382,601 discloses solid pharmaceutical dosage forms containing memantine, which exhibit an extended two-phase release profile, with a portion of the drug being released immediately, followed by a sustained release of the remainder. The matrix of this formulation contains both a water-soluble and a water-insoluble salt of casein, preferably sodium and calcium caseinate. However, casein has an unpleasant taste; it is linked with exacerbation of some side effects as disclosed in U.S. Pat. No. 6,413,556; and displays instability in varying pH. Another concern regarding casein is the possibility of Bovine Spongiform Encephalitis (BSE) contamination since casein is an animal-derived milk protein.

U.S. Pat. No. 6,194,000 discloses a general method of preparing modified release for N-methyl-D-aspartate (NMDA) receptor antagonists. This method involves preparing an immediate release component and a modified release component separately and combining together to arrive at the final formulation. The patent discloses a pellet consisting of a coated core, the coating being any suitable coating using organic solvent-based systems.

U.S. Patent publication No. 20060051416 discloses once-a-day solid oral pharmaceutical dosage forms containing memantine or salt thereof and a pharmaceutically acceptable polymeric carrier (coating and/or matrix) contributing to release a therapeutically effective amount of the active ingredient from about 4 hours to about 24 hours.

U.S. Patent Publication No. 20070065512 discloses a modified and immediate release pharmaceutical dosage forms containing memantine that exhibit an enhanced release profile and provide reliable absorption. The composition disclosed comprises a plurality of beads, each bead comprising an inert core; and a mixture of memantine as an active ingredient and a polymer binder coated on said inert core. Said dosage form exhibits a dissolution rate of active ingredient of about 70% to about 80% within 4 hours to about 24 hours into a use environment.

U.S. Patent publication No. 20060002999 discloses pharmaceutical compositions of 1-aminocyclohexanes, such as memantine or neramexane, prepared by a direct compression method, which are released at a dissolution rate of at least about 80 percent in 60 minutes.

International (PCT) Publication No. WO 2008005036 discloses granulated compositions of memantine.

Hence, even though several approaches have been suggested in the art, it is apparent to one skilled in the art that available technology for effective and reliable extended release, especially multistage release pharmaceutical dosage forms, still leaves a need to provide alternative and improved once a day modified release formulations of memantine with reliable absorption over a targeted period of time and alongside possessing good formulation characteristics desired for bulk manufacturing of the product.

### SUMMARY OF THE INVENTION

In one general aspect there is provided a modified release pharmaceutical composition comprising memantine or salts thereof, one or more pharmaceutically acceptable binders and one or more pharmaceutically acceptable excipients, wherein the composition exhibits a biphasic release profile and the amount of the binder is more than 3% by weight of the composition.

In another general aspect there is provided a modified release pharmaceutical composition comprising coated multiple unit components comprising memantine or salts thereof, one or more pharmaceutically acceptable binders and one or more pharmaceutically acceptable excipients, wherein the composition exhibits a biphasic release profile and the amount of the binder is more than 3% by weight of the composition.

In another general aspect there is provided a modified release pharmaceutical composition comprising:
(a) at least one immediate release component comprising memantine or salts thereof;
(b) a plurality of sustained release components comprising memantine or salts thereof;
(c) one or more pharmaceutically acceptable binders; and
(d) one or more pharmaceutically acceptable excipients,
   wherein the amount of the binder is more than 3% by weight of the composition.

In another general aspect there is provided a modified release pharmaceutical composition comprising:
(a) about 20% by weight of at least one immediate release component comprising memantine or salts thereof;
(b) about 80% by weight of a sustained release component comprising memantine or salts thereof;
(c) one or more pharmaceutically acceptable binders; and
(d) one or more pharmaceutically acceptable excipients,
   wherein the amount of the binder is more than 3% by weight of the composition.

In another general aspect there is provided a modified release pharmaceutical composition of memantine or salts thereof comprising at least one immediate release component exhibiting a dissolution rate of memantine or salts thereof of no less than 10% in 1 hour in simulated gastric fluid, and plurality of sustained release components exhibiting a dissolution rate of memantine or salts thereof of no less than 50% in 6 hour and no less than 80% in 12 hours when measured in simulated gastric fluid using USP Type I dissolution apparatus.

In another general aspect there is provided a modified release pharmaceutical composition comprising matrix of memantine or salts thereof with one or more rate controlling polymers, one or more pharmaceutically acceptable binders and one or more pharmaceutically acceptable excipients, wherein the composition exhibits a biphasic release profile and the amount of the binder is more than 3% by weight of the composition.

In another general aspect there is provided a modified release pharmaceutical composition comprising:
(a) a plurality of sustained release components comprising memantine or salt thereof and one or more rate controlling polymers;
(b) at least one immediate release component comprising memantine or salt thereof coated over said sustained release components; and
(c) more than 3% by weight of one or more pharmaceutically acceptable binders, wherein the composition exhibits a biphasic release profile.

In one aspect, the sustained release components of the modified release pharmaceutical composition comprise a core comprising memantine or salts thereof; an optional barrier layer over the core; and outer one or more layers comprising one or more rate controlling polymers.

In one aspect, the sustained release components comprise more than 3% by weight of one or more pharmaceutically acceptable binders.

In another general aspect, the immediate release components comprise more than 1% by weight of one or more pharmaceutically acceptable binders. In another general aspect there is provided a modified release pharmaceutical composition comprising:
(a) a plurality of sustained release components comprising:
   (i) about 30% to about 80% by weight of water soluble/insoluble inert core;
   (ii) one or more drug layers comprising about 1% to about 15% by weight of memantine or salts thereof coated over the soluble/insoluble inert core;
   (iii) one or more barrier layers comprising about 3.5% to about 15% by weight of hydroxypropyl methylcellulose coated over the drug layer;
   (iv) one or more rate controlling layers comprising about 3% to about 15% by weight of ethyl cellulose coated over the barrier layer; and
(b) at least one immediate release component coated over the sustained release components comprising:
   (i) about 1% to about 5% by weight of memantine or salts thereof; and
   (ii) about 1% to about 6% by weight of hydroxypropyl methylcellulose, wherein the composition exhibits a biphasic release profile.

In another general aspect there is provided a stable modified release pharmaceutical composition comprising memantine or salts thereof, one or more pharmaceutically acceptable binders and one or more pharmaceutically acceptable excipients, wherein the composition retains at least 80% of the potency of the memantine or salts thereof in the pharmaceutical composition after storage for three months at 40° C. and 75% relative humidity, wherein the composition exhibits a biphasic release profile and the amount of the binder is more than 3% by weight of the composition.

In another general aspect there is provided a stable modified release pharmaceutical composition comprising memantine or salts thereof, one or more pharmaceutically acceptable binders and one or more pharmaceutically acceptable excipients, wherein the composition exhibits a biphasic release profile, wherein the amount of the binder is more than 3% by weight of the composition and the excipients comprise a combination of high molecular weight acidic and basic substances.

Embodiments of the modified release pharmaceutical composition may include one or more of the following features. For example, the pharmaceutical composition may further include one or more pharmaceutically acceptable excipients. The pharmaceutically acceptable excipients may include one or more fillers, lubricants, disintegrants, glidants, and the like.

In another general aspect there is provided a process of manufacturing a modified release pharmaceutical composition of memantine or salts thereof, the process comprising:
(a) providing a plurality of sustained release components comprising memantine or salts thereof, one or more rate controlling polymers, one or more pharmaceutically acceptable binders and optionally, one or more pharmaceutical excipients; and
(b) providing at least one immediate release component comprising memantine or salts thereof and one or more pharmaceutical excipients,
   wherein the amount of the binder is more than 3% by weight of the composition.

In another general aspect there is provided a process of manufacturing the modified release pharmaceutical composition of memantine or salts thereof, the process comprising:
(a) providing a matrix and/or a coated core comprising memantine or salts thereof, one or more rate controlling polymers and one or more pharmaceutically acceptable binders;
(b) providing a mixture comprising memantine or salts thereof and one or more pharmaceutically acceptable excipients; and
(c) coating the matrix or the coated core with the mixture,
   wherein the composition exhibits a biphasic release profile and the amount of the binder is more than 3% by weight of the total composition.

In another general aspect there is provided a process of manufacturing the modified release pharmaceutical composition of memantine or salts thereof the process comprising:
(a) providing a plurality of sustained release components comprising memantine or salts thereof;
(b) providing a plurality of immediate release components comprising memantine or salts thereof; and
(c) coating the immediate release components over the sustained release components and formulating the resulting sustained release components into a pharmaceutical dosage form.

Embodiments of the modified release pharmaceutical composition may include one or more of the following features. For example, the pharmaceutical composition may further include one or more pharmaceutically acceptable excipients. The pharmaceutically acceptable excipients may include one or more fillers, lubricants, disintegrants, glidants and the like.

In another general aspect there is provided a method of treating Alzheimer's disease, Parkinson's and neurological diseases, autism, Attention-Deficit/Hyperactivity disorder and autistic spectrum disorders comprising administering to a human patient in need thereof a modified release biphasic pharmaceutical composition comprising memantine or salts thereof, one or more pharmaceutically acceptable binders and one or more pharmaceutically acceptable excipients, wherein the composition exhibits a biphasic release profile and the amount of the binder is more than 3% by weight of the composition.

Embodiments of the pharmaceutical composition may include one or more of the following features. For example, the pharmaceutical composition may further include one or more pharmaceutically acceptable excipients. The pharmaceutically acceptable excipients may include one or more fillers, lubricants, disintegrants, glidants, and the like.

The details of one or more embodiments of then invention are set forth in the description below. Other features, objects and advantages of the invention will be apparent from the description.

### DETAILED DESCRIPTION OF THE INVENTION

The inventors of the present invention have surprisingly found that it is possible to develop a stable and multiple phase release formulation of memantine or salts thereof which is suitable for once daily administration with reliable absorption over a targeted period of time. The formulations according to the present invention also possess good formulation characteristics (such as flowability, compressibility, content uniformity etc) desired for bulk manufacturing of the product.

Multiple phase release formulations involve complex manufacturing process and hence require close monitoring of processing steps, particularly coating and compression of multiple unit components. For example, such multiple unit components are prone to sticking, picking and agglomeration during manufacturing of multiple phase release formulations, which ultimately can affect content uniformity of final dosage forms. The present inventors concomitantly have found that modified release dosage form of memantine or its salt can be formulated into a multiple phase release dosage forms; particularly biphasic release dosage forms using pharmaceutically acceptable excipients in judicial amount which may achieve desired release pattern of memantine from the dosage form.

In particular the inventors have found that if pharmaceutically acceptable binder in amount of more than 3% by weight of the composition is used in the biphasic formulation comprising memantine or salt thereof, the resulting formulation may exhibit excellent formulation characteristics simultaneously providing drug release over 24 hour. Such formulations may also remain stable over the storage period.

Thus, the present invention provides a modified release pharmaceutical composition comprising memantine or salts thereof, one or more pharmaceutically acceptable binders and one or more pharmaceutically acceptable excipients, wherein composition exhibits biphasic release profile and the amount of the binder is more than 3% by weight of the composition. The modified release composition comprises drug containing sustained release components and immediate release components.

The term “biphasic release” as used herein refers to two different phases of release of memantine from the composition, with or without a preceding lag time. The appearance of second phase of release may be detected with a sudden increase in the rate of release at the beginning of the second phase. This can be observed by a change in the slope of the cumulative drug release profile.

The term “modified release pharmaceutical composition” as used hereinbefore and throughout the description includes dosage forms containing combination of components providing immediate release and sustained or controlled or delayed release of memantine or salts thereof from the dosage form.

The term “controlled release” is intended to refer to non-immediate release of memantine or salts thereof from the formulation.

The term “sustained release” is used in its conventional sense to refer to a formulation that provides for gradual release of memantine or salts thereof over an extended period of time, and that preferably, although not necessarily, results in substantially constant blood levels of memantine over an extended time period.

The term “delayed release” is used in its conventional sense to refer to a formulation in which there is a time delay between oral administration of the formulation and the release of memantine or salts thereof. Delayed release may or may not involve gradual release of memantine or salts thereof over an extended period of time, and thus may or may not be sustained release. For example, delayed release formulations of memantine or salts thereof are enterically coated components.

The term “component” as used hereinbefore and throughout the description refers to memantine containing powder, particles, agglomerates, granules, pellets, microspheres, liposomes, sphericles, minitablets, microcapsules, tablets, cores and coats/layers on thereof or any solid physical form known to the person skilled in the art.

The term “memantine” used throughout the specification refers to not only memantine per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. It is also possible to use any salts and free base form of memantine, including polymorphs, hydrates, solvates or amorphous forms. The preferred salt of memantine is hydrochloride salt.

In an embodiment, the amount of memantine or salt thereof in the sustained release components and immediate release components of modified release composition ranges from about 1% to about 15% by weight and about 1% to about 5% by weight respectively.

Suitable “rate controlling polymers” may include one or more of hydrophilic and hydrophobic polymers or mixtures thereof.

Suitable hydrophilic polymers may include one or more of cellulosic polymers/copolymers or its derivatives including methyl cellulose, hydroxypropyl methylcellulose, hydroxyethyl cellulose, hydroxypropyl cellulose, hydroxyethyl methylcellulose, carboxymethylcellulose, sodium carboxymethylcellulose; polyethylene oxides, polyethylene glycols, chitosan, gums, starch derivatives, polyurethanes, galactomannans, polysaccharides, polyalcohols, acrylic acid or acrylamide derivatives and the like. The preferred hydrophilic polymer is hydroxypropyl methylcellulose or any commercially available grade thereof such as Methocel.

Suitable hydrophobic polymers include one or more of ethyl cellulose, glycerol palmitostearate, beeswax, glycowax, carnaubawax, hydrogenated vegetable oil, glycerol monostearate, stearylalcohol, glyceryl behenate, polyanhydrides, methyl acrylates and the like.

The polymers used can also be eroding or non-eroding or combination of both. The polymers, which may be used for bioadhesion, are described below.

Natural polymers include but are not limited to proteins (e.g., hydrophilic proteins), such as pectin, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic; hyaluronic acid, polyhyaluronic acid, alginic acid, sodium alginate.

When the bioadhesive polymer is a synthetic polymer, the synthetic polymer is typically selected from but are not limited to polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers or mixtures thereof.

Other polymers suitable for use in the invention include, but are not limited to, methyl cellulose, ethyl cellulose, hydroxypropyl cellulose, hydroxypropylmethyl cellulose, hydroxybutylmethyl cellulose, cellulose acetate, cellulose propionate, cellulose acetate butyrate, alkyl phthalate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), polyvinyl acetate), polyvinyl chloride, polystyrene, polyvinyl pyrrolidone, and polyvinylphenol. Polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof. Another group of polymers suitable for use as bioadhesive polymers but not necessarily limited to polymers having a hydrophobic backbone with at least one hydrophobic group pendant from the backbone. Suitable hydrophobic groups are groups that are generally non-polar. The amount of rate controlling polymer in the modified release composition ranges from about 3% to about 15% by weight of the composition.

Suitable “pharmaceutically acceptable binders” may include one or more of Methyl cellulose, hydroxyethyl cellulose, hydroxypropyl cellulose, hydroxypropyl methylcellulose, carbomers, dextrin, ethyl cellulose, methylcellulose, shellac, zein, gelatin, gum arabic, polymethacrylates, polyvinyl pyrrolidone, polyvinyl alcohol, polyethylene glycol, carrageenan, polyethylene oxide, waxes, pullulan, agar, tragacanth, veegum, pregelatinized starch, sodium alginate, gums, sugars such as sucrose, maltose, dextrose, lactose, amylase, synthetic resins and the like. The amount of binder for use in the modified release composition is more than 3%, preferably more that 5% by weight of the composition. Preferable pharmaceutically acceptable binder is hydroxypropyl methylcellulose.

It was also surprisingly found that pharmaceutically acceptable binder when used in amount of more that 3% by weight of the sustained release and/or immediate release component, the resulting formulation possess good formulation characteristics.

In an embodiment, the amount of pharmaceutically acceptable binder in the sustained release component and immediate release component in the composition comprises more than 3% by weight and more than I% by weight of composition respectively.

In an embodiment, a stable modified release pharmaceutical composition comprising memantine or salts thereof, one or more pharmaceutically acceptable binder and one or more pharmaceutically acceptable excipients, wherein the composition retains at least 80% of the potency of the memantine or salts thereof in the pharmaceutical composition after storage for three months at 40° C. and 75% relative humidity.

In a further embodiment, a stabilized modified release pharmaceutical composition comprising memantine or salts thereof in accordance with the present invention comprises combination of high molecular weight acidic and basic substances.

The term “acidic and basic substances” as used herein are high molecular weight substances which exhibits acidity and basicity respectively when dissolved or suspended in water. The preferred high molecular weight acidic and basic substances are polymers. However, it will be appreciated to the person skilled in the art that any substance imparting said property can be used as long as it stabilizes the modified release biphasic pharmaceutical composition.

In an embodiment, the sustained release of memantine or salts thereof can be achieved by. providing coated components comprising memantine or salts thereof and one or more rate controlling polymer/s.

In a further embodiment, the modified release pharmaceutical composition comprises one or more sustained release component comprising memantine or salts thereof coated with one or more immediate release component comprising memantine or salts thereof.

In a further embodiment, the modified release pharmaceutical composition comprises memantine or salts thereof and one or more rate controlling polymers, coated with one or more immediate release components comprising memantine or salts thereof.

In a further embodiment, the modified release pharmaceutical composition comprises a core comprising memantine or salts thereof coated with one or more rate controlling polymers, which cores are further coated with one or more immediate release component comprising memantine or salts thereof.

In a further embodiment, sustained release components of the modified release pharmaceutical composition comprise:
(a) a core comprising memantine or salt thereof;
(a) an optional barrier layer over the core; and
(b) an outer one or more layers comprising one or more rate controlling polymers.

In a further embodiment, sustained release components of the modified release pharmaceutical composition comprises water soluble/insoluble inert cores coated by layers of memantine or salt thereof, which is further coated by layers of controlling polymer. The amount of water soluble/insoluble inert core in the composition may range from about 30% to about 80% by weight of the composition.

The modified release pharmaceutical composition may be devised in the form of a tablet, a capsule, granules, pellets, caplets, minitablets, a capsule filled with minitablets and/or pellets, a multi-layer tablet, granules for suspension, or granules filled in a sachet.

In an embodiment, the modified release pharmaceutical composition is not particularly limited as long as it is an oral preparation.

For example, capsules can be packed with one or more tablets, granules or fine granules based on coated sustained release components and immediate release components according to the present invention. Further, hard capsules can be packed with multiple small-diameter mini-tablets based on the sustained release components further coated or compressed with immediate release components of memantine or salts thereof.

In an embodiment, sustained release components are provided in the form of granules, pellets, or minitablets.

The sustained and immediate release components can also be given a barrier/film coating as necessary. It should be noted that the presence or absence of a hydrophilic barrier/film coating on the modified release biphasic preparation according to the present invention has very little effect on the dissolution profile of memantine or salt thereof.

The modified release pharmaceutical composition of memantine or its salt is preferably developed into dosage forms such as coated tablets/granules/pellets or multiple unit particles which can be filled into capsules or compressed to form minitablets or tablets. The composition may be seal coated and finally film coated. The composition can be coated with Ready colour mix systems (such as Opadry colour mix systems).

Alternatively, the modified release pharmaceutical composition of memantine or its salt can be developed using various osmotic-controlled release oral systems (OROS) known in the art.

The modified release pharmaceutical composition of memantine or salts thereof in accordance with the present invention exhibits a dissolution profile which is suitable for once a day dosage regimen.

In particular, the inventors of present invention have surprisingly found that the modified release pharmaceutical composition of memantine or salts thereof comprising pharmaceutically acceptable binder in an amount of more than 3% by weight of the composition, the resulting composition can exhibits no significant difference in rate and/or extent of absorption of memantine as compared to memantine formulation marketed under the trade name Namenda®XR. Advantageously, when sustained release and immediate release components of memantine or salts thereof are employed in an amount of about 80% and 20% by weight of the composition respectively, the resulting formulation achieve drug release over 24 hour.

In an embodiment, the weight ratio of immediate and sustained release components present in the modified release pharmaceutical composition ranges from about 1:0.15 to about 1:0.33.

In an embodiment, the modified release composition in accordance with the present invention, the immediate release component exhibits dissolution rate of memantine or salts thereof of no less than 10% in 1 hour in simulated gastric fluid, and sustained release components exhibit a dissolution rate of memantine or salts thereof of no less than 50% in 6 hour and no less than 80% in 12 hours when measured in simulated gastric fluid using USP Type I dissolution apparatus.

In a further embodiment, the modified release composition in accordance with the present invention, the immediate release component exhibits dissolution rate of memantine or salts thereof of no less than 15% in 1 hour in simulated gastric fluid, and sustained release components exhibits dissolution rate of memantine or salts thereof of no less than 55% in 6 hour and no less than 85% in 12 hours when measured in simulated gastric fluid using USP Type I dissolution apparatus.

The modified release pharmaceutical composition or sustained release and immediate release components therein may be prepared by processes known to the person having ordinary skill in the art of pharmaceutical technology such as direct compression, wet or dry granulation, slugging, hot melt granulation, hot melt extrusion, fluidized bed granulation, extrusion-spheronization, spray drying and solvent evaporation.

In an embodiment, the sustained release component according to the present invention can be prepared by forming coating cores comprising memantine or salts thereof with one or more rate controlling polymers and one or more pharmaceutically acceptable excipients. The sustained release components are further coated with one or more rate controlling polymers.

In a further embodiment, the immediate release components according to the present invention can be prepared in the form of mixture, granules, pellets, coating dispersion by using various methods known the person skilled in the art.

In a further embodiment, the process of manufacturing the modified release pharmaceutical composition of memantine or salts thereof in accordance with the present invention comprises:
(a) providing plurality of coated cores comprising memantine or salts thereof, one or more rate controlling polymers and one or more pharmaceutically acceptable binders;
(b) providing a mixture comprising memantine or salts thereof and one or more pharmaceutically acceptable excipients to provide immediate release of memantine; and
(c) coating the cores prepared in step (a) with the mixture and formulating the resulting cores into a pharmaceutical dosage form.

In a further embodiment, the process of manufacturing the modified release pharmaceutical composition of memantine or salts thereof in accordance with the present invention comprises:
(a) providing a plurality of sustained release components comprising memantine or salts thereof;
(b) providing a plurality of immediate release components comprising memantine or salts thereof; and
(c) coating the immediate release components over the sustained release components and formulating the resulting sustained release components into a pharmaceutical dosage form.

In a further embodiment, the process of manufacturing the modified release pharmaceutical composition of memantine or salts thereof in accordance with the present invention comprises:
(a) providing plurality of sustained release components comprising inert pellets made up of water soluble (e.g. lactose) or water insoluble material (e.g. microcrystalline cellulose) coated with first layer comprising memantine or salts thereof and second layer comprising one or more rate controlling polymers (e.g. hydroxypropyl methylcellulose);
(b) providing a mixture comprising memantine or salts thereof and one or more pharmaceutically acceptable excipients to provide immediate release of memantine,
(c) coating the sustained release components with the mixture and formulating the resulting sustained release components into a pharmaceutical dosage form.

In a further embodiment, the process of manufacturing a modified release pharmaceutical composition of memantine or salts thereof in accordance with the present invention comprises:
(a) coating inert core made up of water soluble (e.g. lactose or sugar) or water insoluble material (e.g. microcrystalline cellulose) with memantine or salts thereof with one or more pharmaceutically acceptable excipient to form drug coated pellets;
(b) providing a film coat over the drug coated pellets;
(c) providing one or more layers of rate controlling polymer over the film coated pellets;
(d) providing a immediate release coating dispersion/solution comprising memantine or salts thereof and one or more pharmaceutically acceptable excipients;
(e) coating the immediate release coating solution/dispersion prepared in step (d) over the pellets prepared in step (c);
(f) optionally, providing a film coat on the pellets prepared in step (e); and
(g) filing the pellets prepared in step (e) or (f) in hard gelatin capsules.

The pharmaceutically acceptable excipients may include one or more fillers, lubricants, disintegrants, glidants, colorants, sweeteners, plasticizers and the like.

Suitable fillers may include, but not limited to one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like.

Suitable disintegrants may include, but not limited to one or more of croscarmellose sodium, sodium starch glycolate, pregelatinized starch, sodium carboxymethyl cellulose, cross-linked polyvinylpyrrolidone and the like.

Suitable plasticizers may include, but not limited to one or more of glycerin fatty acid esters; triethyl citrate; propylene glycol; polyethylene glycol and the like.

Suitable lubricants and glidants may include one or more of talc, metallic stearates such as magnesium stearate, calcium stearate, zinc stearate; colloidal silicon dioxide, finely divided silicon dioxide, stearic acid,.hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like

Suitable plasticizers may include, but not limited to one or more of polyols such as glycerol, propylene glycol, polyethylene glycol (PEG), urea, or other known plasticizers such as triethyl citrate, dibutyl or dimethyl phthalate or water.

Suitable examples of colorants include, but not limited to one or more of non-water soluble lake pigments; neutral pigments; yellow ferric oxide; red ferric oxide; black iron oxide and the like.

In a further embodiment, the invention provides a method of treating Alzheimer's disease, Parkinson's and neurological diseases, autism, Attention-Deficit/Hyperactivity disorder and autistic spectrum disorders comprises administering to a human patient in need thereof a modified release pharmaceutical composition comprising memantine or salts thereof, one or more pharmaceutically acceptable binder and one or more pharmaceutically acceptable excipients.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

TABLE 1

| | | |
|---|---|---|
| Sr. No. | Ingredients/Strength | Mg/Capsule |

Drug Loading

| | | |
|---|---|---|
| 1 | Memantine Hydrochloride | 22.400 |
| 2 | Sugar spheres | 120.000 |
| 3 | Hydroxypropyl methylcellulose | 11.200 |
| 4 | Talc | 2.40 |
| 5 | Isopropyl alcohol | q.s. |
| 6 | Purified water | q.s. |

Seal Coating

| | | |
|---|---|---|
| 1 | Hydroxypropyl methylcellulose | 5.000 |
| 2 | Triethyl citrate | 0.500 |
| 3 | Talc | 1.000 |
| 4 | Purified water | q.s. |

Sustained release coating

| | | |
|---|---|---|
| 1 | Ethyl cellulose | 20.000 |
| 2 | Hydroxypropyl methylcellulose | 2.500 |
| 3 | Triethyl citrate | 2.250 |
| 4 | Talc | 0.250 |
| 5 | Isopropyl alcohol | q.s. |
| 6 | Methylene chloride | q.s. |

Immediate release coating

| | | |
|---|---|---|
| 1 | Memantine Hydrochloride | 5.600 |
| 2 | Hydroxypropyl methylcellulose | 2.800 |
| 4 | Talc | 0.600 |
| 5 | Isopropyl alcohol | q.s. |
| 6 | Purified water | q.s. |

Top coating

| | | |
|---|---|---|
| 1 | Hydroxypropyl methylcellulose | 5.000 |
| 2 | Triethyl citrate | 0.500 |
| 3 | Talc | 1.000 |
| 4 | Purified water | q.s. |

Lubrication

| | | |
|---|---|---|
| 1 | Talc | 1.000 |
| | Total weight | 204.000 |

Procedure: Sugar spheres were coated with drug solution containing memantine hydrochloride, purified water, isopropyl alcohol, hydroxypropyl methylcellulose and talc. The drug coated pellets were then seal coated with aqueous dispersion containing hydroxypropyl methylcellulose, triethyl citrate, talc and purified water. A further sustained release coat containing ethyl cellulose, hydroxypropyl methylcellulose triethyl citrate, talc, isopropyl alcohol and methylene chloride was then applied on the seal coated pellets. Separately, memantine hydrochloride was dissolved in isopropyl alcohol, hydroxypropyl methylcellulose, talc and purified water to form immediate release coating solution of the drug which was then applied over the sustained release coat of the pellets. The pellets were then coated with film coating solution containing hydroxypropyl methylcellulose, triethyl citrate, talc and purified water. Coated pellets were lubricated with talc and finally filled into capsules.

TABLE 2

| | % Drug |
|---|---|
| Time (hr) | Dissolved |
| 0 | 0 |
| 1 | 11 |
| 2.5 | 27 |
| 4 | 39 |
| 6 | 57 |
| 8 | 77 |
| 12 | 94 |
| 16 | 98 |

Table 2 provides dissolution data for memantine HCl tablet prepared as per Example 1. For determination of drug release rate, USP Type I apparatus (100 rpm) was used wherein 900 ml of water and HCl/NaCl buffer of 1.2 pH was used as medium.

TABLE 3

Test
Related Substances

| | Any individual | Total degradation | |
|---|---|---|---|
| Duration | degradation product | products | |
| (Month) | NMT 0.2% | NMT 1.0% | Assay |
| Initial | ND | ND | 99.9 |
| 1 month | BQL | BQL | 99.4 |
| 2 month | BQL | BQL | 98.6 |
| 3 month | BQL | BQL | 99.2 |
| 6 month | BQL | BQL | 98.7 |

ND: Not Detected
BQL: Below Quantitation Limit

Table 3 provides stability data for memantine HCl tablet prepared as per Example 1 when stored at 40° C. and 75% relative humidity for six months and indicates that the composition remains stable over the storage period.

While the invention has been described in terms of its specific embodiments, certain modifications and equivalents will be apparent to those skilled in the art and are intended to be included within the scope of the invention.

We claim:

1. A modified release pharmaceutical composition comprising:
(a) a plurality of sustained release components comprising memantine or salts thereof and one or more rate controlling polymers;
(b) at least one immediate release component comprising memantine or salts thereof coated over the sustained release components; and
(c) more than 3% by weight of one or more pharmaceutically acceptable binders, wherein the composition exhibits a biphasic release profile.

2. The modified release pharmaceutical composition as claimed in claim 1, wherein the sustained release components comprise:
(a) a core comprising memantine or salts thereof;
(a) an optional barrier layer over the core; and
(b) an outer one or more layers comprising one or more rate controlling polymers.

3. The modified release pharmaceutical composition as claimed in claim 2, wherein the core comprises water soluble/insoluble inert cores coated with memantine or salts thereof.

4. The modified release pharmaceutical composition as claimed in claim 1, wherein the rate controlling polymer comprises one or more of ethyl cellulose, glycerol palmitostearate, beeswax, glycowax, carnaubawax, hydrogenated vegetable oil, glycerol monostearate, stearylalcohol, glyceryl behenate, polyanhydrides, methyl acrylates, alkyl phthalates, cellulose acetate phthalate, polyethylene oxides, and polyethylene glycols.

5. The modified release pharmaceutical composition as claimed in claim 1, wherein the pharmaceutically acceptable binder comprises one or more of methyl cellulose, hydroxyethyl cellulose, hydroxypropyl cellulose, hydroxypropyl methylcellulose, carbomers, dextrin, ethyl cellulose, methylcellulose, shellac, zein, gelatin, gum arabic, polymethacrylates, polyvinyl pyrrolidone, polyvinyl alcohol, polyethylene glycol, carrageenan, polyethylene oxide, waxes, pullulan, agar, tragacanth, veegum, pregelatinized starch, sodium alginate, gums and sugars.

6. The modified release pharmaceutical composition as claimed in claim 1, wherein the sustained release components comprise more than 3% by weight of one or more pharmaceutically acceptable binders.

7. The modified release pharmaceutical composition as claimed in claim 1, wherein the immediate release components comprise more than 1% by weight of one or more pharmaceutically acceptable binders.

8. The modified release pharmaceutical composition as claimed in claim 1, wherein the sustained release components are in the form of granules, pellets, or minitablets.

9. The modified release pharmaceutical composition as claimed in claim 1 in the form of a tablet, a capsule, granules, pellets, caplets, minitablets, a capsule filled with minitablets and/or pellets, a multi-layer tablet, granules for suspension, or granules filled in a sachet.

10. The modified release pharmaceutical composition as claimed in claim 1, wherein the composition comprises about 20% by weight of the immediate release component, and about 80% by weight of the sustained release components.

11. The modified release pharmaceutical composition as claimed in claim 1, wherein the immediate release component exhibits a dissolution rate of memantine or salts thereof of no less than 10% in 1 hour in simulated gastric fluid, and the sustained release components exhibit a dissolution rate of memantine or salts thereof of no less than 50% in 6 hour and no less than 80% in 12 hours when measured in simulated gastric fluid using USP Type dissolution apparatus.

12. The modified release pharmaceutical composition as claimed in claim 1, wherein the composition retains at least 80% of the potency of memantine or salts thereof in the composition after storage for three months at 40° C. and 75% relative humidity.

13. The modified release pharmaceutical composition as claimed in claim 1, wherein the composition further comprises a combination of high molecular weight acidic and basic substances.

14. A modified release pharmaceutical composition comprising:
(a) a plurality of sustained release components comprising:
(i) about 30% to about 80% by weight of water soluble/insoluble inert core;
(ii) one or more drug layers comprising about 1% to about 15% by weight of memantine or salts thereof coated over soluble/insoluble inert core;
(iii) one or more barrier layers comprising about 3.5% to about 15% by weight of hydroxypropyl methylcellulose coated over the drug layer; and
(iv) one or more rate controlling layers comprising about 3% to about 15% by weight of ethyl cellulose coated over the barrier layer; and
(b) at least one immediate release component coated over the sustained release components comprising:
(i) about 1% to about 5% by weight of memantine or salts thereof, and
(ii) about 1% to about 6% by weight of hydroxypropyl methylcellulose, wherein the composition exhibits a biphasic release profile.

15. A process for the preparation of a modified release pharmaceutical composition, the process comprising:
(a) providing a plurality of sustained release components comprising memantine or salts thereof;
(b) providing a plurality of immediate release components comprising memantine or salts thereof; and
(c) coating the immediate release components over the sustained release components and formulating the resulting sustained release components into a pharmaceutical dosage form.

16. The process as claimed in claim 15, wherein the sustained release components are prepared by a process comprising:
(a) coating water soluble/insoluble inert cores with one or more layers comprising memantine or salts thereof and optionally one or more pharmaceutically acceptable excipients to provide memantine coated cores;
(b) optionally, coating a barrier layer over the memantine coated cores; and
(b) coating at least one layer comprising one or more rate controlling polymers over the memantine coated cores or the barrier layer.

17. The process as claimed in claim 15, wherein the immediate release components are prepared by mixing memantine or salts thereof with one or more pharmaceutically acceptable binders and one or more pharmaceutically acceptable excipients.

18. The process as claimed in claim 15, wherein the composition comprises about 20% by weight of the immediate release components and about 80% by weight of the sustained release components.

19. A method of treating Alzheimer's disease, Parkinson's and neurological diseases, autism, Attention-Deficit/Hyperactivity disorder and autistic spectrum disorders, the method comprising administering to a human patient in need thereof the modified release pharmaceutical composition as claimed in claim 1.
